(19) United States
(12) Patent Application Publication
(10) Pub. No.: US 2026/0325506 A1
(43) Pub. Date: Oct. 22, 2026

(54) METHODS OF TREATING POST-TRAUMATIC STRESS DISORDER AND ALCOHOL USE DISORDER

(71) Applicants: FLORIDA ATLANTIC UNIVERSITY RESEARCH CORPORATION, Boca Raton, GA (US); THE BOARD OF REGENTS OF THE UNIVERSITY OF OKLAHOMA, Norman, OK (US)

(72) Inventors: Lawrence TOLL, Palm City, FL (US); Andrea CIPPITELLI, West Palm Beach, FL (US); Kelly STANDIFER, Norman, OK (US)

(73) Assignees: FLORIDA ATLANTIC UNIVERSITY RESEARCH CORPORATION, Boca Raton, GA (US); THE BOARD OF REGENTS OF THE UNIVERSITY OF OKLAHOMA, Norman, OK (US)

(21) Appl. No.: 19/485,481

(22) PCT Filed: May 17, 2024
(86) PCT No.: PCT/US2024/029885

§ 371 Date: Nov. 18, 2025

Related U.S. Application Data
(60) Provisional application No. 63/502,950, filed on May 18, 2023.

(51) Int. Cl. A61K 31/485; A61K 31/135; A61K 31/145; A61K 31/16; A61K 31/197; A61K 31/4525; A61K 31/7048; A61K 45/06; A61P 25/22

(52) U.S. Cl. CPC A61K 31/485; A61K 31/135; A61K 31/145; A61K 31/16; A61K 31/197; A61K 31/4525; A61K 31/7048; A61K 45/06; A61P 25/22

(57) ABSTRACT

Disclosed herein are compositions and methods for the treatment of comorbid post-traumatic stress disorder and alcohol use disorder.

### CROSS-REFERENCE TO RELATED APPLICATION

This application claims the benefit of U.S. Provisional Application 63/502,950, filed May 18, 2023, the contents of which are hereby incorporated in its entirety.

### FIELD OF THE INVENTION

Disclosed herein are compositions and methods for the treatment of comorbid post-traumatic stress disorder and alcohol use disorder.

### BACKGROUND

Traumatic stress encompasses a broad spectrum of consequences to trauma exposure. Post-traumatic stress disorder (PTSD) is a disordered physiological and psychological response to severe trauma that is not necessarily associated with physical injury or even experiencing the trauma oneself; witnessing a traumatic event may be sufficient. Considerable overlap exists between PTSD, traumatic brain injury (TBI) and alcohol abuse disorder (AUD) patient cohorts, with 63% of veterans with PTSD also diagnosed with AUD. Similar numbers are noted for current service members and the public at large. These conditions all commonly include irritability, pain, depression, anxiety, emotional numbing, avoidance, cognitive impairment, insomnia, fatigue, and hyperarousal. Both clinical and preclinical studies show that PTSD and TBI are obvious and important risk factors for the development of substance use disorders, including AUD, and AUD predisposes one to PTSD (and TBI). Meta-analysis found that exposure to a traumatic memory generated a significantly greater increase in alcohol craving than exposure to alcohol-related or neutral cues. While the FDA has approved only two drugs to treat PTSD and three to treat AUD, there are no FDA-approved treatments for PTSD and co-morbid AUD. Treatment is further complicated in patients with comorbid pain. Existing therapies for PTSD and AUD are often ineffective and fraught with adverse effects and drug-drug interactions, leading to poor patient compliance. These factors underscore the urgent need to identify and develop potential therapeutic targets for these increasingly prevalent conditions.
PPL-138 (14β-(3-phenylpropanoyl)-17-cyclopropylmethyl-7,8-dihydronoroxymorphinone) has the formula:

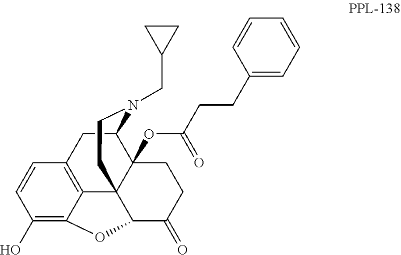

PPL-138 has high affinity (Ki less than 10 nM) at all four members of the opioid receptor family (mu, kappa, delta, NOP), a very low efficacy at mu (less than buprenorphine), partial agonist activity at NOP receptor (higher than buprenorphine) and is an antagonist at kappa and delta receptors (like buprenorphine).

There remains a need for improved systems and methods for treating PTSD. There remains a need for improved systems and methods for treating AUD. There remains a need for improved systems and methods for treating PTSD comorbid with AUD.

### BRIEF DESCRIPTION OF THE FIGURES

FIG. 1 depicts how PPL-138 reduces SPS induced anxiety like behaviors. PPL-138 treatment (1 mg/kg, i.p.) reduced the SPS increased anxiety index (A) and increased open arm time (B) and open arm entries (C). PPL 138+SPS differed from SPS as determined by Tukey's post hoc test with *p<0.05 and **p<0.01 (GraphPad Prism v9.4). Scatter plot of mean±S.D.

FIG. 2 depicts how SPS reduces paw withdrawal threshold, consistent with allodynia, and increases thermal sensitivity which was reversed with PPL 138.

FIG. 3 depicts how PPL-138 reduces anxiety-like behaviors in male (FIG. 3A) and female (FIG. 3B) Sprague Dawley (SD) rats.

FIG. 4 depicts how PPL-138 reduces allodynia and hyperalgesia in SD rats.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Disclosed herein are methods of treating a patient diagnosed with post-traumatic stress disorder and alcohol use disorder, by administering to the patient a pharmaceutical composition including PPL-138, i.e., the compound:

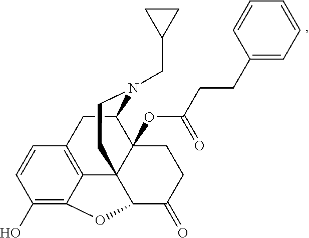

or a pharmaceutically acceptable salt thereof.

Also disclosed are methods of treating alcohol use disorder in a patient diagnosed with post-traumatic stress disorder, by administering to the patient a pharmaceutical composition including PPL-138, i.e., the compound:

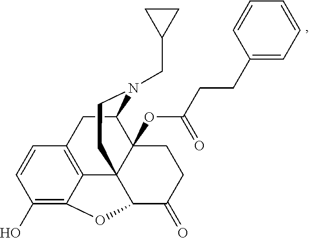

or a pharmaceutically acceptable salt thereof.

Also disclosed are methods of promoting alcohol use cessation in a patient diagnosed with post-traumatic stress disorder, by administering to the patient a pharmaceutical composition including PPL-138, i.e., the compound:

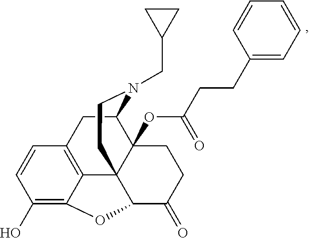

or a pharmaceutically acceptable salt thereof.

Also disclosed are methods of reducing alcohol self-administration in a patient diagnosed with post-traumatic stress disorder by administering to the patient a pharmaceutical composition including PPL-138, i.e., the compound:

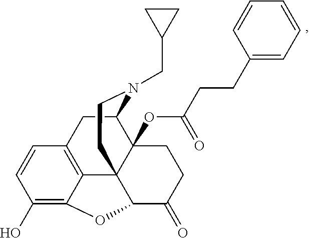

or a pharmaceutically acceptable salt thereof.

In certain implementations, PPL-138 can be used to treat alcohol dependence. In some implementations PPL-138 can be used to reduce alcohol cravings.

The patient can be diagnosed with acute post-traumatic stress disorder or with chronic post-traumatic stress disorder. In some implementations the patient may have additional co-morbidities, for example traumatic brain injury, allodynia, or hyperalgesia. In certain implementations the patients can be diagnosed with diabetes, fibromyalgia, shingles, trigeminal neuralgia, complex regional pain syndrome, amplified musculoskeletal pain syndrome, or multiple sclerosis. In other implementations the patient will not be diagnosed with any additional conditions apart from PTSD and alcohol use disorder.

PPL-138 can be used to treat individual symptoms of PTSD and/or alcohol use disorder. For example PPL-138 can be used to reduce any one or more of irritability, pain, depression, anxiety, emotional numbing, avoidance, cognitive impairment, insomnia, fatigue, or hyperarousal. In certain implementations PPL-138 can be used to reduce the occurrence and/or severity of traumatic memories.

PPL-138 can be used to treat a variety of different human patients. In some implementations the patient is an adult patient, a pediatric patient (<18 years of age), or a geriatric patient (>64 years of age). In some implementations the patient is male, and in certain implementations the patient is female. In some implementations the patient is pregnant.

PPL-138 can be used to treat patients in a variety of settings. In some implementations, PPL-138 can be used to treat a patient admitted to a healthcare facility. In some implementations PPL-138 can be used to treat patients in an out-patient setting (i.e., not admitted to a healthcare facility). In some implementations the patient can receive supportive counseling while being treated with PPL-138.

In certain implementations, PPL-138 can be administered as the free base, while in some implementations PPL-138 can be formulated as a salt. Reference to PPL-138 without further qualification is inclusive of the free base, salts, and combinations thereof. In certain implementations PPL-138 can be formulated as the hydrochloride salt, the hydrobromide salt, the sulfate salt, the hydrogen sulfate salt, the maleate salt, the tartrate salt, the succinate salt, or the acetate salt.

PPL-138 can be administered in a therapeutically effective amount. In certain implementations PPL-138 can be administered in an amount from 0.001-500 mg, 0.001-1 mg, 0.001-0.1 mg, 0.001-0.01 mg, 0.1-1 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, 50-100 mg, 100-250 mg, or 250-500 mg. Unless specified to the contrary, the dosage amounts described herein refer to the amount of PPL-138 free base.

PPL-138 can be administered by various routes. In some implementations, PPL-138 can be administered by a route selected from inhalational, oral, nasal, rectal, parenteral, sublingual, transdermal, transmucosal intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, topical administration, and combinations thereof. In certain implementations PPL-138 can be administered orally as part of an ingestible dosage form or sublingually.

PPL-138 can be administered using a particular dosing schedule. For example, PPL-138 can be administered 4 times a day, 3 times a day, twice a day, once a day, once every two days, once every three days, once every five days, once every seven days, or once every ten days.

PPL-138 can be administered in combination with at least one additional opioid antagonist, partial antagonists, partial agonist, or combination thereof. In certain implementations, PPL-138 can be administered in combination with naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, and combination thereof.

PPL-138 can be administered in combination with one or more additional alcohol use disorder therapies. In some implementations PPL-138 is administered in combination with an alcohol dehydrogenase inhibitor, NMDA antagonist, anticonvulsant, antidepressant, anxiolytic, or combination thereof. Exemplary alcohol dehydrogenase inhibitors include disulfiram, exemplary NMDA antagonists include acamprosate, exemplary anticonvulsants include gabapentin, pregabalin, and topiramate. In some combinations PPL-138 is administered in combination with a benzodiazepine. Exemplary benzodiazepines include alprazolam, bromazepam, brotizolam, chlordiazepoxide, clobazam, clonazepam, clorazepate, diazepam, estazolam, flunitrazepam, flurazepam, flutoprazepam, halazepam, ketazolam, loprazolam, lorazepam, lormetazepam, midazolam, nimetazepam, nitrazepam, oxazepam, prazepam, quazepam, temazepam, and triazolam.

In some implementations PPL-138 is administered in combination with a antidepressant, for example an SSRI. In some implementations, PPL-138 is administered in combination with sertraline or paroxetine.

### EXAMPLES

The following examples are for the purpose of illustration of the invention only and are not intended to limit the scope of the present invention in any manner whatsoever.

SPS procedure: 2 h of complete restraint in disposable plastic holders, followed by grouped (2-3 rats) forced swim for 20 min in a cylindrical tank (46 cm tall×20 cm in diameter) filled with 22-25° C. water to a depth of 30 cm. After 15 min recovery and drying period, rats are exposed to diethyl ether until loss of consciousness and left undisturbed (except for drug or vehicle injection) and alone for 7 days, after which they are pair housed. All rats are housed with soft bedding, observed for signs of pain or discomfort: weight loss, lack of grooming, or porphyrin staining; any such rats is excluded from study.

Elevated Plus Maze (EPM): Rats will be tested for anxiety-like behaviors. The percentage of open arm entries (number of entries into the open arms divided by total number of entries in both arms), time spent in the open arms, total distance traveled, and total time spent immobile are noted. The anxiety index (AI) is calculated where total exploration on the maze represents the total number of arm entries: Anxiety Index=1−[[(time spent in open arms/total time on the maze (300 sec))+(number of entries into open arms/total number of arm entries)]/2]. Each animal is tested at 9 and 30 days post-SPS.

Drug Treatment: PPL-138 (0.3 and 1 mg/kg) is dissolved in the same vehicle as 10 mg/kg SB (3% DMSO+97% of 0.5% hydroxypropyl cellulose (HPC)). Drug treatment (i.p.) begins immediately after exposure to ether and continues every other day thereafter. Ctr and SPS rats receive vehicle (3% DMSO+97% 0.5% HPC) injections.

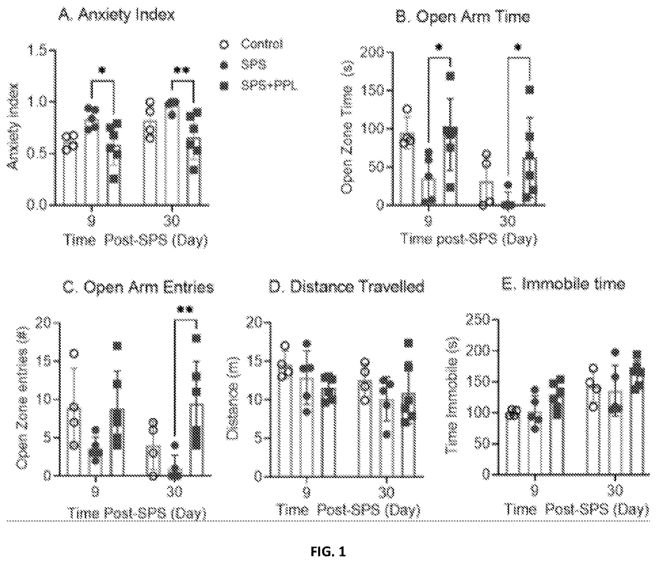

FIG. 1 demonstrates that anxiety-like behaviors in male Sprague Dawley (SD) rats are evident at day 9 as determined by elevated plus maze (EPM) and are maintained or exacerbated at day 30. Anxiety index reflects a combination of three anxiety-like behaviors. Two-way ANOVA with repeated measures found significant effects of treatment for anxiety index [F (2,12)=9.912; p<0.005], Open arm time [F (2,12)=5.066; p<0.05], and open arm entries [F (2,12)=11.99; p<0.005], and significant effect of PPL-138 (1 mg/kg) (FIG. 1A-C). Significant effects of time were noted for anxiety index [(F1,12)=5.466; p<0.05], open arm time [F (1,12)=12.86; p<0.01] and immobile time [F1, 12)=29.9; p<0.001]; no differences were noted between groups in distance travelled.

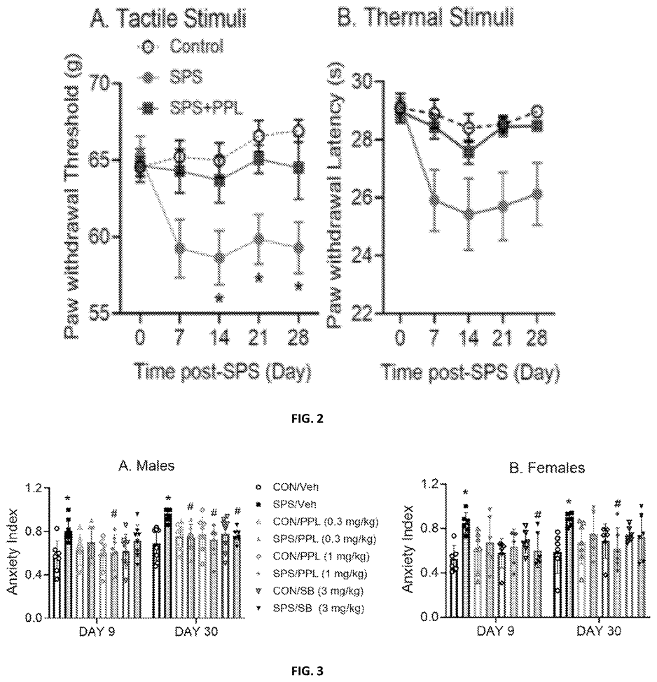

Degree of enhanced nociceptive sensitivity by SPS to tactile and thermal stimuli were reduced in this cohort likely a result of group housing during the first 7 days instead of our usual solo housing (FIG. 2). There was a significant effect of treatment for sensitivity to both tactile (A) and thermal (B) stimuli ([F (2, 12)=8.938, p<0.005] and [F (2, 12)=7.56, p<0.01]), respectively, as well as a significant effect of time for thermal sensitivity [F (4, 48)=5.147, p<0.01]. Tukey's multiple comparisons test indicates that SPS rats had significantly lower paw withdrawal threshold on days 14, 21 and 28 than Ctr or SPS+PPL-138 (1 mg/kg) rats (FIG. 2A), p<0.05). One-way ANOVA of serum samples from all rats obtained at end of the pilot study showed a significant difference in N/OFQ levels between treatment groups [F (2, 12)=4.147; p=0.0421]. PPL-138 reduced serum N/OFQ levels compared to SPS (p<0.05 by Tukey's post-hoc test).

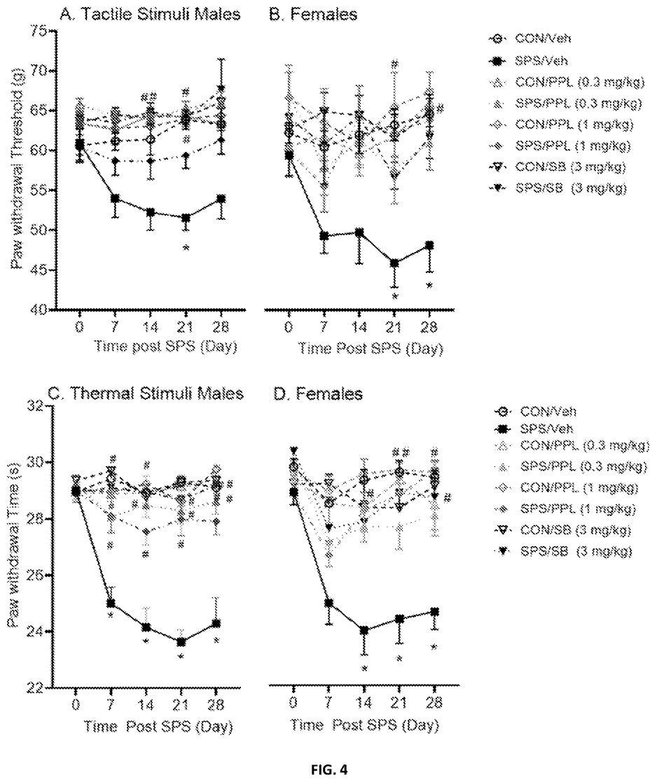

Control (Ctr) and SPS-treated male and female SD rats are treated 3 times each week with Veh, PPL-138 (0.3 and 1 mg/kg, s.c.) or the NOP antagonist SB612111 (SB; 3 mg/kg, i.p.). The efficacy of the partial agonist is compared its respective control group for anxiolysis (FIG. 3) and antinociception (FIG. 4); the antagonist, SB, is included as a positive control for the males. SB alleviates TBI- and 6-OHDA Parkinson disease model-induced locomotor deficits and brain hypoxia. SB also produces antinociception in models of neuropathy and irritable bowel syndrome (IBS). SB behaves similarly to a less selective NOP receptor antagonist, JTC-801, that reversed SPS-induced allodynia and anxiety-like behaviors.

FIG. 3 demonstrates that anxiety-like behaviors in male (FIG. 3A) and female (FIG. 3B) Sprague Dawley (SD) rats are evident from the Anxiety index at day 9 and day 30 as determined by elevated plus maze (EPM). In males, two-way ANOVA with repeated measures found significant effects of treatment [F (7, 57)=4.347; p=0.0006] and time [F (1, 57)=41.69; p<0.0001], with significant effect of PPL-138 (1 mg/kg) at day 9, and significant effects of 0.3 and 1 mg/kg of PPL-138 and of SB at day 30 post-SPS (FIG. 3A). The results from females are not yet fully powered (n=6 instead of n=8 for males), but two-way ANOVA with repeated measures found a significant effect of treatment [F (3, 42)=7.444; p=0.0004] with significant effect of SB at day 9, and significant effect of PPL-138 (1 mg/kg) at day 30 post-SPS (FIG. 3B).

Sensitivity to tactile and thermal sensitivity in males and females was determined by paw withdrawal threshold to increasing pressure (PWT; tactile) and paw withdrawal latency to a thermal stimulus (PWL) (FIG. 4). There was a significant interaction between time and treatment for sensitivity to both tactile (A) and thermal (C) stimuli for males ([F (28, 228)=1.708, p=0.0182] and [F (28, 228)=4.276, p<0.0001]), respectively, as well as for thermal sensitivity (D) in females [F (28, 164)=2.286, p=0.0007]. There was a significant effect of treatment on tactile sensitivity in females (B) [F (7,41)=7.060; p<0.0001]. Tukey's multiple comparisons test indicates that male and female SPS rats developed significant allodynia (A, B) and thermal hyperalgesia (C, D) over 28 days compared to vehicle controls. Allodynia was reversed by 0.3 mg/kg PPL-138 and by SB in males, and by 1 mg/kg PPL-138 in females (#p<0.05). Thermal hyperalgesia was reversed by both doses of PPL-138 and SB in males, and by 1 mg/kg PPL-138 and SB in females.

The data demonstrate that administration of the partial agonist reverses development of PTSD-induced nociceptive sensitivity and anxiety-like behaviors in males and females.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches

1. A method of treating a patient diagnosed with post-traumatic stress disorder and alcohol use disorder, the method comprising administering to the patient a pharmaceutical composition comprising the compound: 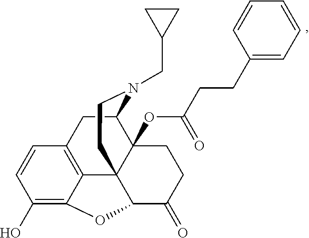
or a pharmaceutically acceptable salt thereof.

2. A method of treating alcohol use disorder in a patient diagnosed with post-traumatic stress disorder, the method comprising administering to the patient a pharmaceutical composition comprising the compound: 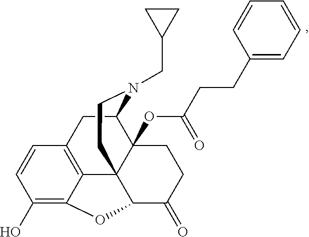
or a pharmaceutically acceptable salt thereof.

3. A method of promoting alcohol use cessation and/or reducing alcohol self-administration in a patient diagnosed with post-traumatic stress disorder, the method comprising administering to the patient a pharmaceutical composition comprising the compound: 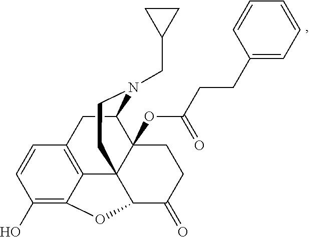
or a pharmaceutically acceptable salt thereof.

4. (canceled)

5. The method of claim 1, wherein the alcohol use disorder is alcohol dependence.

6. The method of claim 1, wherein the method is for reducing alcohol cravings.

7. The method of claim 1, wherein the patient is diagnosed with acute post-traumatic stress disorder and/or chronic post-traumatic stress disorder.

8. (canceled)

9. The method of claim 1, wherein the method is for reducing irritability, pain, depression, anxiety, emotional numbing, avoidance, cognitive impairment, insomnia, fatigue, hyperarousal, or a combination thereof.

10. The method of claim 1, wherein the method is for reducing the production of traumatic memories.

11. (canceled)

12. The method of claim 1, wherein the patient is admitted to an in-patient healthcare facility and/or treated as an out-patient.

13. (canceled)

14. The method of claim 1, wherein the patient is a pediatric patient, an adult patient, or a geriatric patient (≥65 years of age).

15-16. (canceled)

17. The method of claim 1, wherein the compound is the hydrochloride salt, the hydrobromide salt, the sulfate salt, the hydrogen sulfate salt, the maleate salt, the tartrate salt, the succinate salt, or the acetate salt.

18. The method of claim 1, wherein the compound is administered at a dose of 0.001-500 mg.

19. The method of claim 1, wherein the composition is administered by a route selected from inhalational, oral, nasal, rectal, parenteral, sublingual, transdermal, transmucosal intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, topical administration, and combinations thereof.

20. The method of claim 1, wherein the composition is administered 4 times a day, 3 times a day, twice a day, once a day, once every two days, once every three days, once every five days, once every seven days, or once every ten days.

21. The method of claim 1, further comprising administering at least one additional opioid antagonist, partial opioid antagonist, partial opioid agonist, or a combination thereof.

22. The method of claim 1, further comprising administering naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, or a combination thereof.

23. The method of claim 1, further comprising administering an anxiolytic, alcohol dehydrogenase inhibitor, NMDA antagonist, anticonvulsant, antidepressant, other serotonergic agent, or a combination thereof.

24. The method of claim 23, wherein the alcohol dehydrogenase inhibitor is disulfiram, the NMDA antagonist is acamprosate, and/or the anticonvulsant is gabapentin, pregabalin, or topiramate.

25. The method of claim 1, further comprising administering sertraline HCl or paroxetine HCl to the patient.

26. The method of claim 1, wherein the patient receives supportive counseling.
